(19) United States
(12) Patent Application Publication
(10) Pub. No.: US 2026/0207148 A1
(43) Pub. Date: Jul. 23, 2026

(54) PUNCTURE GUIDANCE METHOD, FAST LOW-DOSE CT PUNCTURE GUIDANCE METHOD, AND TRIPLE-SOURCE COMPUTED TOMOGRAPHY SYSTEM

(71) Applicant: BEIJING PHOTON COUNTING TECHNOLOGY LTD., Beijing (CN)

(72) Inventors: Zhanfeng XING, Beijing (CN); Xin WANG, Beijing (CN); Miao YU, Beijing (CN); Weiqiang ZHAO, Beijing (CN); Wanfeng LI, Beijing (CN); Na GAO, Beijing (CN); Hao XU, Beijing (CN)

(21) Appl. No.: 19/137,493

(22) PCT Filed: Nov. 20, 2024
(86) PCT No.: PCT/CN2024/133254

§ 371 Date: Jun. 10, 2025

(30) Foreign Application Priority Data
Sep. 25, 2023 (CN) 202311243175.0

(51) Int. Cl. A61B 6/12; A61B 6/03; A61B 6/40; A61B 6/42; A61B 17/34; A61B 90/00

(52) U.S. Cl. CPC A61B 6/12; A61B 6/03; A61B 6/4007; A61B 6/4266; A61B 17/34; A61B 90/37; A61B 2090/3762

(57) ABSTRACT

A puncture guidance method, a fast low-dose computed tomography (CT) puncture guidance method, and a triple-source CT system. The puncture guidance method includes: performing, in a triple-source CT system, a full scan or half scan to obtain a first image, wherein the first image corresponds to a puncture needle being at a first position; determining, in an image domain, a plurality of second images according to the first image, wherein the second images correspond to the puncture needle being at other positions different from the first position; and determining a position of the puncture needle by using actual projection data from triple-source and triple-detector at three preset angles and forward projections of the plurality of second images at the three angles.

### CROSS-REFERENCE TO RELATED APPLICATIONS

The present disclosure is based on and claims the priority to the Chinese application No. 202311243175.0 filed on Sep. 25, 2023, the disclosure of which is incorporated herein in its entirety.

### TECHNICAL FIELD

The present disclosure relates to the application field of medical X-ray imaging equipment, and specifically to a puncture guidance method, a fast low-dose CT puncture guidance method, and a triple-source computed tomography system.

### BACKGROUND

Computed Tomography (CT) guided interventional surgery is an interventional technique of percutaneous punctures into non-blood-vessels with effective intensions, includes a CT guided percutaneous puncture biopsy and interventional therapy, and is usable in various parts of the whole body, including head, chest, abdomen, musculoskeletal and other systems.

In the CT guided interventional surgery, since by means of CT images, a lesion part is accurately positioned and tissues and blood vessels around the lesion can be clearly known, a needle insertion position, angle and depth can be accurately determined, and can be adjusted at any time under CT scanning and monitoring, thereby achieving accurate puncture.

The CT guided interventional surgery can also be combined with a “robot”, to complete a puncture plan by three-dimensional CT images, and guide a robotic arm to complete the interventional surgery in a planned route.

Common CT guided interventional surgery includes: a neuralgia interventional therapy, pulmonary nodule puncture biopsy, abdominal or liver abscess drainage, etc.

In related CT guided puncture operations, there are problems such as slow scan speed, and a high radiation dose to a patient.

At present, an operation specification has been formed for CT guided interventional operations, and the interventional & minimally invasive committee of China Medicine Education Association and the interventional branch of Chinese Medical Doctor Association have jointly published “CT GUIDED INTERVENTIONAL OPERATION SPECIFICATION, 2022 EDITION” (JOURNAL OF MEDICAL IMAGING, 2022, vol. 32, no. 3, interventional & minimally invasive committee of China Medicine Education Association, interventional branch of Chinese Medical Doctor Association).

For CT guided interventional operations in conventional single/dual source CT, low-dose CT “Fluoro” is typically used for guidance. There are mainly two types of Fluoro scan modes, where one is an intermittent exposure mode, i.e. completing one full scan over 360-degree or half scan over 180-degree plus fan-angle for every exposure, needle insertion and exposure being performed alternately; the other is a continuous exposure mode, i.e. a CT device performing continuous exposure and continuous image reconstruction, exposure and needle insertion being performed simultaneously. However, in both modes, exposure over at least 180 degrees plus a fan angle is required to obtain a current position of a needle, and in a current system with a fastest gantry rotation speed (0.25 s/rotation), exposure for about 0.16 s is required, which is a relatively slow speed (unable to implement real-time display) and has a relatively high radiation dose.

### SUMMARY

In the present disclosure, in order to solve the defects and shortcomings in the related art described above, there is provided a fast low-dose CT puncture guidance method capable of completing puncture guidance by a few scans and several pulsed exposure data acquisitions, with a fast speed of feedback to needle insertion and a great reduction in radiation dose in a puncture process.

A technical solution adopted in the present disclosure for solving the technical problem thereof is: a puncture guidance method, comprising: performing, in a triple-source computed tomography (CT) system, a full scan or half scan to obtain a first image, wherein the first image corresponds to a puncture needle being at a first position; determining, in an image domain, a plurality of second images according to the first image, wherein the second images corresponds to the puncture needle being at other positions different from the first position; and determining a position of the puncture needle by using actual projection data from triple-source and triple-detector at preset three angles and forward projections of the plurality of second images at the three angles.

In some embodiments, the determining, in an image domain, a plurality of second images according to the first image, comprises: determining an image of the puncture needle according to the first image; and determining the plurality of second images according to the image of the puncture needle and the first image, by gradually displacing the puncture needle along a needle insertion direction in the image domain.

In some embodiments, the determining an image of the puncture needle according to the first image, comprises: placing a patient in an appropriate posture and scan position for a scan to obtain an original image before the puncture needle insertion; and obtaining the image of the puncture needle by performing image subtraction between the first image and the original image.

In some embodiments, the puncture guidance method further comprises: obtaining a direction of the puncture needle by a line detection method according to the image of the puncture needle, wherein the needle insertion direction is along the direction of the puncture needle into a patient.

In some embodiments, the determining a position of the puncture needle by using actual projection data of triple-source and triple-detector at preset three angles and forward projections of the plurality of second images at the three angles, comprises: performing forward projection at the three angles for each second image, according to geometric arrangement of the triple-source computed tomography system and the preset three angles; subtracting the forward projections of each second image from the actual projection data for the corresponding angles, to determine differences between the forward projections of each second image and the actual projection data for the corresponding angles; determining, among the plurality of second images, a second image with a minimum sum of absolute values of the differences or a minimum sum of squares of the differences; and determining the position of the puncture needle according to the second image with the minimum sum of the absolute values of the differences or the minimum sum of the squares of the differences.

In some embodiments, the determining the position of the puncture needle according to the second image with the minimum sum of the absolute values of the differences or the minimum sum of the squares of the differences, comprises: determining the position corresponding to the second image as the position of the puncture needle, in a case where the sum of the absolute values of the differences corresponding to the second image or the sum of the squares of the differences is less than a threshold; and re-performing the obtaining the first image, the determining the plurality of second images according to the first image, and the determining the position of the puncture needle according to actual projection data and forward projections of the plurality of second images, in a case where the sum of the absolute values of the differences corresponding to the second image or the sum of the squares of the differences is greater than or equal to the threshold.

In some embodiments, the puncture guidance method further comprises: stopping rotation of a gantry of the triple-source computed tomography system, so that three tubes in the triple-source computed tomography system stop at the preset angles, and performing exposure to acquire the actual projection data for the three angles; or keeping the rotation of the gantry of the triple-source computed tomography system, and performing exposure at specific angles to acquire the actual projection data for the three angles.

In some embodiments, the first position is a position where a depth of the puncture needle reaches a preset depth threshold.

In some embodiments, the full scan is a scan with exposure over 360 degrees; and the half scan is a scan with exposure over an angle being a sum of a fan angle of the triple-source and triple-detector and 180 degrees.

In some embodiments, the gradually displacing, in the image domain, gradually displacing the puncture needle along a needle insertion direction in the image domain comprises: keeping the puncture needle displacing along a same straight line, with an interval between displacements not greater than ½ pixel on each coordinate axis.

In some embodiments, the exposure is pulsed exposure or exposure controlled by milliampere modulation.

In some embodiments, the puncture guidance method further comprises: performing verification by using two-dimensional data and using a numerical simulation method.

According to another aspect of the present disclosure, there is provided A fast low-dose CT puncture guidance method, comprising: calculating a change in a position of a puncture needle by using projection data from triple-source and triple-detector at preset three angles (a gantry is rotating or is static), after a scan over one circle or over 180 degrees plus a fan angle is completed in a triple-source CT system; determining whether a calculation result is reliable, by a consistency between forward projections for the three angles and the actual projection data, thereby completing puncture guidance by one scan and several pulsed exposures, with fast scan speed and a greatly reduced radiation dose.

In some embodiments, specific implementation steps of the fast low-dose CT puncture guidance method are as follows:
1) placing a patient in an appropriate posture and scan position for a scan to obtain original data and image (Img₀) before the needle insertion;
2) inserting the needle according to a pre-planned needle insertion direction and path;
3) acquiring data over one circle or over 180 degrees plus a fan angle in a rotation manner after the needle insertion is started (the needle is located at a certain depth inside the patient), and reconstructing an image (Img₁);
4) obtaining an image of the needle (Img_{needle}) by performing image subtraction between the images (Img₁−Img₀);
5) obtaining a direction of the (straight) needle by a line detection method, wherein the needle insertion direction is along the direction of the puncture needle into the patient;
6) gradually displacing the needle along the needle insertion direction in an image domain while keeping the needle on a same straight line, with an interval between displacements not greater than ½ pixel on each coordinate axis, to form a series of image data ImgCN₁ . . . ImgCNₙ;
7) stopping rotation of a gantry, so that three tubes stop at the preset angles; or keeping the rotation of the gantry, and performing exposure at specific angles to acquire data;
8) performing pulsed exposure as needed;
9) acquiring projection data proj1ₙ, proj2ₙ and proj3ₙ for the three angles corresponding to three combinations of tube and detector for each pulsed exposure;
10) performing forward projection in three directions on ImgCNₓ according to geometrical arrangement of the system and the preset tube angles in the step 7), for the data obtained in the step 6), and subtracting the forward projection from the real projection data for the corresponding angles obtained in the steps 8) and 9)
11) calculating differences, performing thresholding processing on the differences, calculating and summing absolute values or summing the squares, to find ImgCNₘᵢₙ with a minimum sum of the absolute values of the differences or a minimum sum of the squares of the differences, and in a case where the sum of the absolute values of the differences or the sum of the squares of the differences corresponding to the ImgCNₘᵢₙ is less than a threshold TH, considering that the needle insertion depth and the image after the needle insertion are approximate to the ImgCNₘᵢₙ, and using the ImgCNₘᵢₙ as the image after the needle insertion for display;
12) considering that the needle insertion path deviates from expectation in a case where the sum of the absolute values of the differences or the sum of the squares of the differences corresponding to the ImgCNₘᵢₙ is greater than the threshold TH, for example, there is a rotation or translation in the needle insertion path, or the patient has a displacement, and at this time, prompting an operator to perform one 360-degree full scan or half scan to obtain Imgᵣ; and
13) obtaining a new image before the needle insertion Imgᵣ₀ by performing rigid registration (rotation+translation) between the image before the needle insertion Img₀ and Imgᵣ, and continuing, from the step 4), calculation and display of the needle insertion process.

In some embodiments, in the step 7), a tube 1 is stopped at 0 degree; a tube 2 is stopped at 240 degrees; and a tube 3 is stopped at 120 degrees. However, the angles of the 3 tubes when stopped have no influence on the feasibility of the method, and can be any angle, but an angle of an interval between the tubes is determined by pre-design of the system. Meanwhile, stopping the rotation of the gantry is not necessary, by pulsed exposure at specific angles in the rotation process, the same effect can be achieved, and an interval for the pulsed exposure is selected according to a requirement of a refresh rate.

In some embodiments, milliampere modulation with a larger amplitude may be used instead of the pulsed exposure in a case where X-ray source hardware does not support the pulsed exposure in the step 8), to greatly reduce the dose.

In some embodiments, the algorithm and processes of this method are verified by using a numerical simulation method, in which two-dimensional data is used, but the method itself is not limited to the two-dimensional data.

According to yet another aspect of the present disclosure, there is provided a triple-source computed tomography system configured to perform the puncture guidance method as described above or the fast low-dose CT puncture guidance method as described above.

In the present disclosure, puncture guidance can be completed by a few scans and several pulsed radiations, with fast scan speed, which can track the puncture and needle insertion in real time, and meanwhile, greatly reduce the radiation dose.

### BRIEF DESCRIPTION OF THE DRAWINGS

FIG. 1 is a composition diagram of a CT system used in the present disclosure;

FIG. 2A is a schematic flow diagram according to some embodiments of the present disclosure;

FIG. 2B is a schematic flow diagram according to other embodiments of the present disclosure;

FIG. 3 is a schematic diagram of simulated use of an XCAT digital motif according to the present disclosure;

FIG. 4 is a schematic diagram of a high-density straight line segment simulating a needle for puncture according to the present disclosure;

FIG. 5 is an image after needle insertion according to the present disclosure;

FIG. 6 is one position in a series of images ImgCNₘ which can be obtained by displacing a needle in a needle insertion direction according to the present disclosure;

FIG. 7 is a curve plot of a sum of absolute values of differences for a certain needle insertion depth and other positions according to the present disclosure;

FIG. 8 is a curved surface plot of a sum of absolute values of differences between three projections for all needle insertion positions and other needle insertion positions according to the present disclosure;

FIG. 9 is a curved surface plot of a sum of absolute values of differences between projections when a patient and a needle are rotated together by 0.5 degree according to the present disclosure;

FIG. 10 is a curved surface plot of a sum of absolute values of differences between projections of a needle and positions in an ideal needle insertion path when the needle is rotated by 0.5 degree for deep insertion in the present disclosure;

FIG. 11 is a corresponding curved surface plot when there is a one-pixel offset of a needle from an ideal needle insertion path in the present disclosure;

FIG. 12 is an image after noise is added according to the present disclosure;

FIG. 13 is a schematic diagram of comparison before and after thresholding processing according to the present disclosure;

FIG. 14 is a three-dimensional curved surface plot of differences for all needle insertion depths according to the present disclosure;

FIG. 15 is a schematic diagram after thresholding processing is performed and noise is added according to the present disclosure.

### DETAILED DESCRIPTION

The technical solutions in the embodiments of the present disclosure will be clearly and completely described below in conjunction with the drawings in the embodiments of the present disclosure, and it is obvious that the described embodiments are only some of the embodiments of the present disclosure, rather than all of the embodiments. All other embodiments, which can be obtained by one of ordinary skill in the art based on the embodiments in the present disclosure without making any creative labor, shall fall within the scope of protection of the present disclosure.

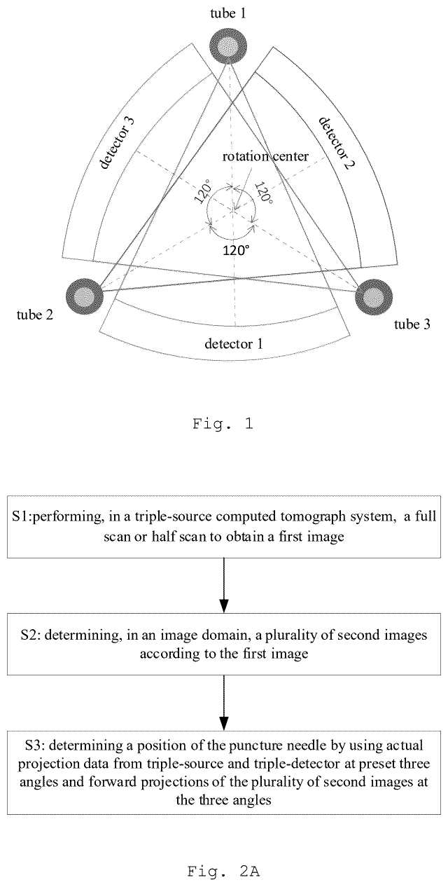

As shown in FIG. 1, according to the present disclosure, in a triple-source computed tomography (CT) system, after a scan over one circle or over 180 degrees plus a fan angle is completed, a change in a position of a puncture needle is calculated by using projection data of triple-source and triple-detector at preset three angles (a gantry is rotating or is static), and it is determined, by a consistency between forward projections for the three angles and the actual projection data, whether a calculation result is reliable. Therefore, according to this apparatus and the method, puncture surgery can be completed by one scan and several pulsed radiations, with fast scan speed and a greatly reduced radiation dose.

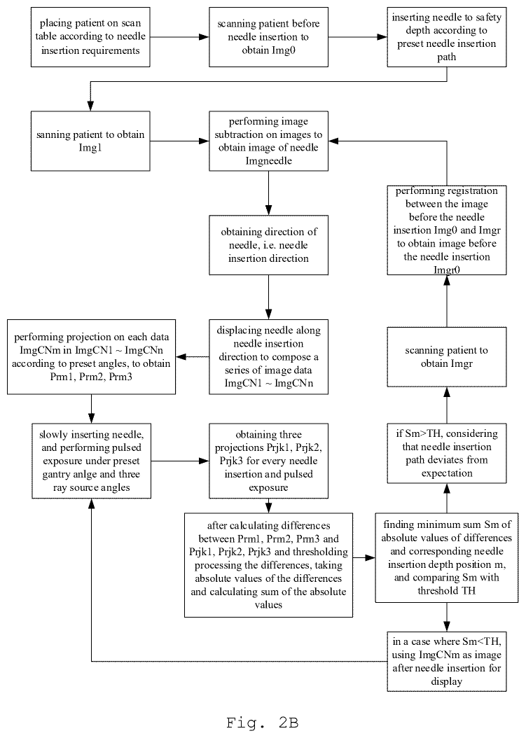

First, a basic implementation of the present disclosure will be described in conjunction with FIG. 2A. In some embodiments, a puncture guidance method comprises: step S1, performing, in a triple-source CT system, a full scan or half scan to obtain a first image, wherein the first image corresponds to a puncture needle being at a first position; step S2, determining, in an image domain, a plurality of second images according to the first image, wherein the second images corresponds to the puncture needle being at other positions different from the first position; and step S3, determining a position of the puncture needle by using actual projection data from triple-source and triple-detector at preset three angles and forward projections of the plurality of second images at the three angles.

A specific embodiment of the puncture guidance method provided in the present disclosure will be described below in conjunction with FIG. 2B. As shown in FIG. 2B, specific implementation steps of the present disclosure are as follows:
1) placing a patient in an appropriate posture and scan position for a scan to obtain original data and image (Img₀) before the needle insertion, in other words, to obtain an original image without the puncture needle;
2) inserting the needle according to a pre-planned needle insertion direction and path;
3) acquiring data over one circle or over 180 degrees plus a fan angle in a rotation manner after the needle insertion is started (the needle is located at a certain depth inside the patient), and reconstructing an image (Img₁), i.e. a first image, wherein the first image corresponds to the puncture needle being at the certain depth inside the patient;
4) obtaining an image of the needle (Img_{needle}) by performing image subtraction between the images (Img₁−Img₀);
5) obtaining a direction of the (straight) needle by a line detection method, wherein the needle insertion direction is along the direction of the puncture needle into the patient;
6) gradually displacing the needle along the needle insertion direction in an image domain while keeping the needle on a same straight line, with an interval between displacements not greater than ½ pixel on each coordinate axis, to form a series of image data ImgCN₁ . . . ImgCNₙ, i.e. second images, wherein the second images correspond to the puncture needle being at other positions;
7) stopping rotation of a gantry, so that three tubes stop at the preset angles; or keeping the rotation of the gantry, and performing exposure at specific angles to acquire data (for example, a tube 1 is at 0 degree; a tube 2 is at 240 degrees; and a tube 3 is at 120 degrees);
8) performing pulsed exposure as needed (milliampere modulation with a larger amplitude can be used instead of the pulsed exposure in a case where X-ray source hardware does not support the pulsed exposure, to greatly reduce a dose, i.e., the exposure is controlled by the milliampere modulation with the larger amplitude);
9) acquiring actual projection data proj1ₙ, proj2ₙ and proj3ₙ for the three angles for every pulsed exposure;
10) for the data (the plurality of second images) obtained in the step 6), performing forward projection in three directions on each second image ImgCNₓ according to geometrical arrangement of the system and the preset tube angles in the step 7), and subtracting it from the real projection data for the corresponding angles obtained in the steps 8) and 9);
11) calculating differences, performing thresholding processing on the differences, calculating and summing absolute values or summing the squares, to find ImgCNₘᵢₙ with a minimum sum of the absolute values of the differences or a minimum sum of the squares of the differences, and in a case where the sum of the absolute values of the differences or the sum of the squares of the differences corresponding to the ImgCNₘᵢₙ is less than a threshold TH, considering that the needle insertion depth and the image after the needle insertion are approximate to the ImgCNₘᵢₙ, and using the ImgCNₘᵢₙ as the image after the needle insertion for display;
12) considering that the needle insertion path deviates from expectation in a case where the sum of the absolute values of the differences or the sum of the squares of the differences corresponding to the ImgCNₘᵢₙ is greater than the threshold TH, for example, there is a rotation or translation in the needle insertion path, or the patient has a displacement, and at this time, prompting an operator to perform one 360-degree full scan or half scan to obtain Imgᵣ;
13) obtaining a new image before the needle insertion Imgᵣ₀ by performing rigid registration (rotation+translation) between the image before the needle insertion Img₀ and Imgᵣ, and continuing, from the step 4), calculation and display of the needle insertion process, comprising re-performing the three steps of obtaining a first image, determining a plurality of second images according to the first image, and determining a position of the needle according to actual projection data and forward projections of the plurality of second images.

In some embodiments, the present disclosure further provides a triple-source computed tomography (CT) system, capable of performing the puncture guidance method according to any of the above embodiments, so that puncture surgery can be completed by one scan and several pulsed radiations, with fast scan speed and a greatly reduced radiation dose.

In the present disclosure, the algorithm and processes are verified by using a numerical simulation method, in which single-slice data is used for the verification, but the method itself is not limited to the single-slice data.

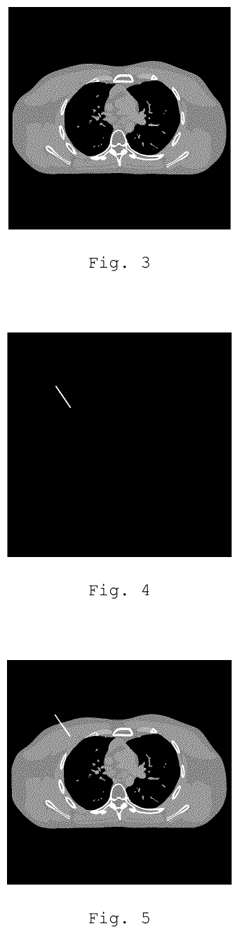

A certain digital motif, e.g., XCAT (see FIG. 3), is used for simulation.

As shown in FIG. 4, a high-density straight line segment simulates a needle for puncture.

An image after needle insertion is shown in FIG. 5.

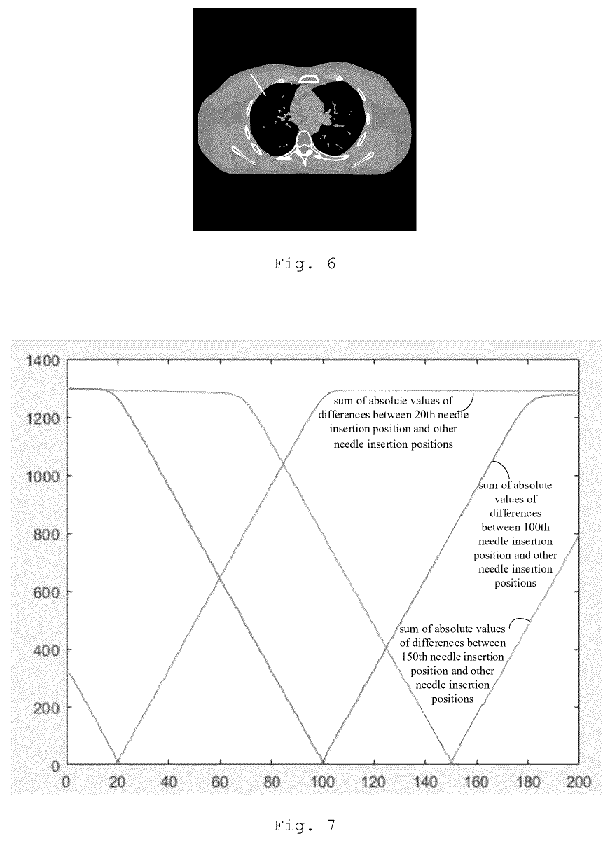

The needle is displaced in a needle insertion direction, and a series of images imgCNₘ can be obtained, one position therein being shown in FIG. 6.

A sum of absolute values of differences for a certain needle insertion depth and other positions can be displayed as one curve (as shown in FIG. 7); and a sum of absolute values of differences between three projections for all needle insertion positions and other needle insertion positions can be displayed as one curved surface. By the overall data analysis, it can be found that, regardless of any position at which the needle is located, a needle insertion position and a corresponding composite image can be obtained by finding a minimum of a sum of absolute values of differences between three projections for this position and three projections corresponding to different needle insertion depths.

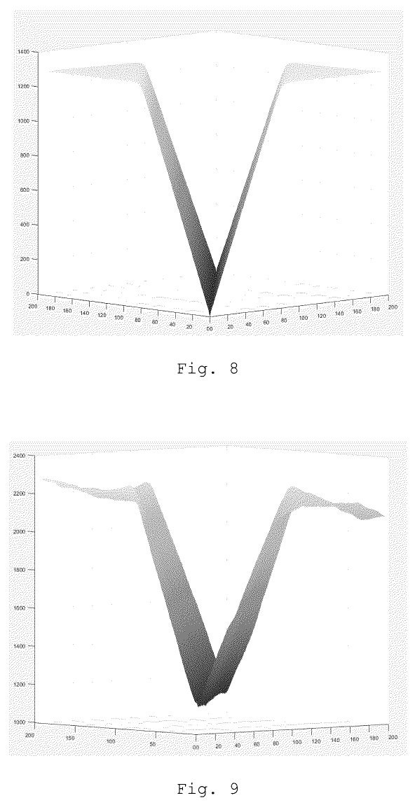

In FIG. 8, x and y coordinates are a current needle position and another needle insertion position, respectively, and z is a sum of absolute values of differences between three projections corresponding to the current needle insertion position and the other needle insertion position. FIG. 8 shows that, without considering noise, a sum of absolute values of differences between three projections corresponding to the current needle insertion position and an forward projection of a composite image closest to the current needle insertion position is approximately 0. A minimum of a sum of absolute values of differences between an forward projection of a composite image with a difference of one pixel and three projections for the current position is 25.8.

When the patient and the needle are rotated together by 0.5 degree, although a minimum of a sum of absolute values of differences can still be found for each needle insertion depth, the minimum is much greater than a sum absolute values of differences between projections when there are only different needle insertion depths. FIG. 9 shows a curved surface of a sum of absolute values of differences between projections when the patient and the needle are rotated together by 0.5 degree. A minimum of a sum of absolute values of differences for all positions is 1131.8.

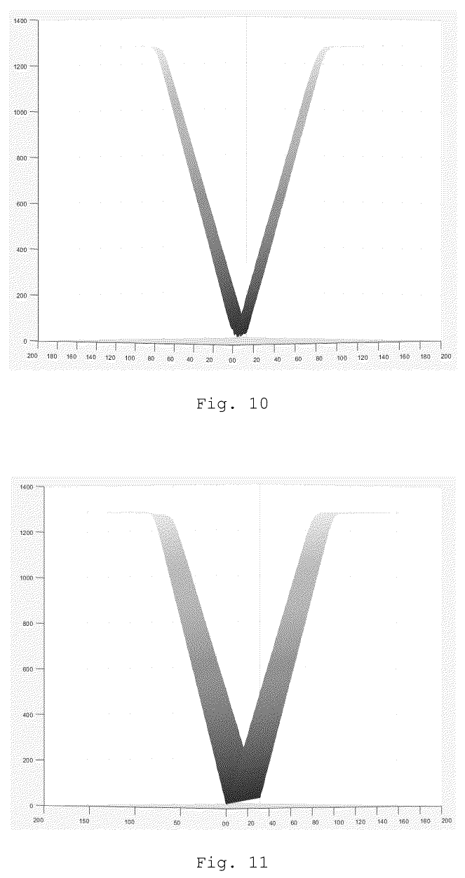

When only the needle does not follow a needle insertion path for deep insertion, but is rotated by 0.5 degree for deep insertion, a curved surface of a sum of absolute values of differences between projections for the needle and positions of an ideal needle insertion path is shown in FIG. 10. A minimum of a sum of absolute values of differences for all the positions is 19.2.

When there is a one-pixel offset of the needle from the ideal needle insertion path (because the needle insertion path is determined after first needle insertion, this situation theoretically should not occur), a corresponding curved surface is shown in FIG. 11. A minimum of a sum of absolute values of differences for positions with which needle insertion depths are most matched (positions with a minimum sum of absolute values of differences) is 18.8.

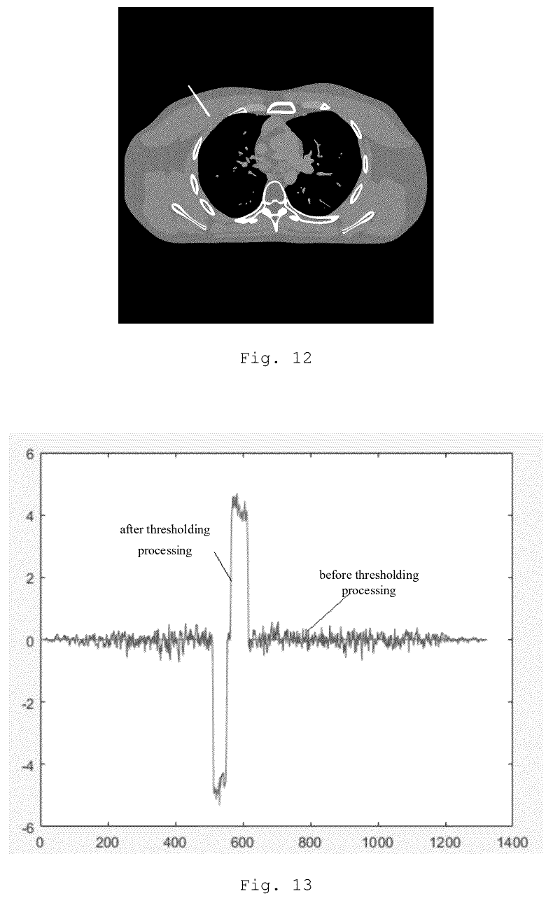

When there is noise in the image, in order to simulate a lower dose, an Standard Deviation (SD) value of 20 is selected, and an image after noise is added is shown in FIG. 12.

When there is the noise, if the noise is not processed, it will result in a large sum of absolute values of differences. Therefore, it is necessary to perform thresholding processing on the difference. In this example, a threshold of 1.1 is selected (as shown in FIG. 13), which can perfectly filter the noise, and will not influence the change in the projection value caused by the difference of the position of the needle.

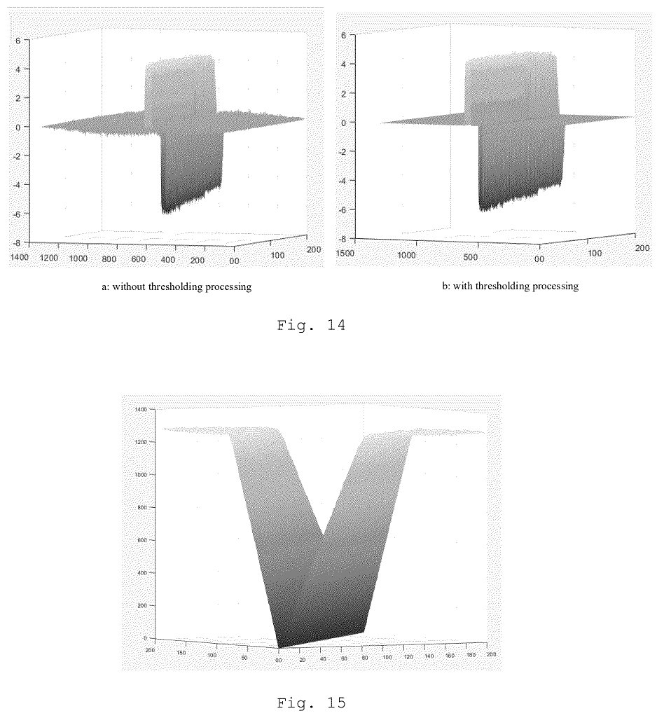

Differences for all the needle insertion depths can also be displayed as a three-dimensional curved surface, as shown in FIG. 14.

After the thresholding processing, for the image after the noise with the SD of 20 is added, a minimum of a sum of absolute values of differences between projections at various needle insertion positions and the actual position is significantly lower than that in a case where there is rotational or translational position deviation, specifically as shown in FIG. 15. A minimum of a sum of absolute values of differences for all the positions is 0, and a maximum thereof is 1.2. By taking a threshold TH between 1.2 and 18.8, it is possible to track the position of the needle when the patient or needle does not displace, and prompt to complete one 360-degree scan when the patient or needle has displaced.

So far, various embodiments of the present disclosure have been described in detail. Some details well known in the art have not been described in order to avoid obscuring the concepts of the present disclosure. Those skilled in the art can fully appreciate how to implement the technical solutions disclosed here, in view of the foregoing description.

Although some specific embodiments of the present disclosure have been described in detail by examples, it should be understood by those skilled in the art that the above examples are for illustration only and are not intended to limit the scope of the present disclosure. It should be understood by those skilled in the art that modifications may be made to the above embodiments and equivalent substitutions may be made to part of technical features without departing from the scope and spirit of the present disclosure. The scope of the present disclosure is defined by the appended claims.

1. A puncture guidance method, comprising:
performing, in a triple-source computed tomography (CT) system, a full scan or half scan to obtain a first image, wherein the first image corresponds to a puncture needle being at a first position;
determining, in an image domain, a plurality of second images according to the first image, wherein the plurality of second images correspond to the puncture needle being at other positions different from the first position; and
determining a position of the puncture needle by using actual projection data from triple-source and triple-detector at three preset angles and forward projections of the plurality of second images at the three preset angles.

2. The puncture guidance method according to claim 1, wherein the determining, in the image domain, the plurality of second images according to the first image, comprises:
determining an image of the puncture needle according to the first image; and
determining the plurality of second images according to the image of the puncture needle and the first image, by gradually displacing the puncture needle along a needle insertion direction in the image domain.

3. The puncture guidance method according to claim 2, wherein the determining the image of the puncture needle according to the first image, comprises:
placing a patient in a posture and scan position for a scan to obtain an original image before puncture needle insertion; and
obtaining the image of the puncture needle by performing image subtraction between the first image and the original image.

4. The puncture guidance method according to claim 2, further comprising:
obtaining a direction of the puncture needle by a line detection method according to the image of the puncture needle, wherein the needle insertion direction is along the direction of the puncture needle into a patient.

5. The puncture guidance method according to claim 1, wherein the determining the position of the puncture needle by using the actual projection data of triple-source and triple-detector at the three preset angles and the forward projections of the plurality of second images at the three preset angles, comprises:
performing forward projection at the three preset angles for each second image, according to geometric arrangement of the triple-source CT system and the three preset angles;
subtracting the forward projections of each second image from the actual projection data for corresponding angles, to determine differences between the forward projections of each second image and the actual projection data for the corresponding angles;
determining, among the plurality of second images, a second image with a minimum sum of absolute values of the differences or a minimum sum of squares of the differences; and
determining the position of the puncture needle according to the second image with the minimum sum of the absolute values of the differences or the minimum sum of the squares of the differences.

6. The puncture guidance method according to claim 5, wherein the determining the position of the puncture needle according to the second image with the minimum sum of the absolute values of the differences or the minimum sum of the squares of the differences, comprises:
determining the position corresponding to the second image as the position of the puncture needle, in a case where a sum of the absolute values of the differences corresponding to the second image or the sum of the squares of the differences is less than a threshold; and
re-performing the obtaining the first image, the determining the plurality of second images according to the first image, and the determining the position of the puncture needle according to actual projection data and forward projections of the plurality of second images, in a case where the sum of the absolute values of the differences corresponding to the second image or the sum of the squares of the differences is greater than or equal to the threshold.

7. The puncture guidance method according to claim 1, further comprising:
stopping rotation of a gantry of the triple-source CT system, so that three tubes in the triple-source CT system stop at the three preset angles, and performing an exposure to acquire the actual projection data for the three preset angles; or
keeping the rotation of the gantry of the triple-source CT system, and performing the exposure at specific angles to acquire the actual projection data for the three preset angles.

8. The puncture guidance method according to claim 1, wherein the first position is a position where a depth of the puncture needle reaches a preset depth threshold.

9. The puncture guidance method according to claim 1, wherein:
the full scan is a scan with exposure over 360 degrees; and
the half scan is a scan with exposure over an angle being a sum of a fan angle of the triple-source and triple-detector and 180 degrees.

10. The puncture guidance method according to claim 2, wherein gradually displacing the puncture needle along the needle insertion direction in the image domain comprises:
keeping the puncture needle displacing along a same straight line, with an interval between displacements not greater than ½ pixel on each coordinate axis.

11. The puncture guidance method according to claim 7, wherein the exposure is a pulsed exposure or an exposure controlled by milliampere modulation.

12. The puncture guidance method according to claim 1, further comprising:
performing verification by using two-dimensional data and using a numerical simulation method.

13. A fast low-dose computed tomography (CT) puncture guidance method, comprising:
calculating a change in a position of a puncture needle by using projection data from triple-source and triple-detector at three preset angles, after a scan over one circle or over 180 degrees plus a fan angle is completed in a triple-source CT system; and
determining whether a calculation result is reliable, based on a consistency between forward projections for the three preset angles and actual projection data, thereby completing puncture guidance by one scan and several pulsed exposures, with fast scan speed and a greatly reduced radiation dose.

14. The fast low-dose CT puncture guidance method according to claim 13, wherein specific implementation steps are as follows:
1) placing a patient in a posture and scan position for a scan to obtain original data and a first image before needle insertion;
2) inserting a needle according to a pre-planned needle insertion direction and a pre-planned needle path;
3) acquiring data over the one circle or over the 180 degrees plus a fan angle in a rotation manner after the needle insertion is started, and reconstructing a second image after the needle insertion;
4) obtaining an image of the needle by performing image subtraction of the first image from the second image;
5) obtaining a direction of the needle by a line detection method, wherein the needle insertion direction is along the direction of the puncture needle into the patient;
6) gradually displacing the needle along the needle insertion direction in an image domain while keeping the needle on a same straight line, with an interval between displacements not greater than ½ pixel on each coordinate axis, to form a series of image data;
7) stopping rotation of a gantry, so that three tubes stop at the three preset angles; or keeping the rotation of the gantry, and performing exposure at specific angles to acquire data;
8) performing pulsed exposure;
9) acquiring projection data for the three preset angles corresponding to three combinations of tube and detector for each pulsed exposure;
10) performing forward projection in three directions on the series of image data according to geometrical arrangement of step 7), for the data obtained in step 6), and subtracting the forward projection from real projection data for corresponding angles obtained in steps 8) and 9);
11) calculating differences, performing thresholding processing on the differences, calculating and summing absolute values or summing squares, to find a minimum difference image data with a minimum sum of the absolute values of the differences or a minimum sum of the squares of the differences, and considering that needle insertion depth and the image after the needle insertion are approximate to the minimum difference image data in a case where a sum of the absolute values of the differences or a sum of the squares of the differences corresponding to the minimum difference image data is less than a threshold, and using the minimum difference image data as the image after the needle insertion for display;
12) considering that the needle insertion path deviates from expectation in a case where the sum of the absolute values of the differences or the sum of the squares of the differences corresponding to the minimum difference image data is greater than the threshold, for example, there is a rotation or translation in the needle insertion path, or the patient has a displacement, and at this time, prompting an operator to perform one 360-degree full scan or half scan to obtain a registration image; and
13) obtaining a new image before the needle insertion by performing rigid registration between the first image, and the registration image, continuing, from step 4), calculation and display of the needle insertion process.

15. The fast low-dose CT puncture guidance method according to claim 14, wherein in step 7), positions of the three tubes are known, and arrangement of a plurality of tubes is known.

16. The fast low-dose CT puncture guidance method according to claim 14, wherein in step 8), milliampere modulation with a larger amplitude is used instead of the pulsed exposure to greatly reduce a dose, in a case where X-ray source hardware does not support the pulsed exposure.

17. The fast low-dose CT puncture guidance method according to claim 14, wherein an algorithm and processes of this method are verified by using a numerical simulation method, in which two-dimensional data is used.

18. A triple-source computed tomography (CT) system configured to perform a puncture guidance, comprising:
performing, in a triple-source CT system, a full scan or half scan to obtain a first image, wherein the first image corresponds to a puncture needle being at a first position;
determining, in an image domain, a plurality of second images according to the first image. wherein the second images correspond to the puncture needle being at other positions different from the first position; and
determining a position of the puncture needle by using actual projection data from triple-source and triple-detector at three preset angles and forward projections of the plurality of second images at the three preset angles.

19. A triple-source CT system configured to perform the fast low-dose CT puncture guidance method according to claim 13.

20. The fast low-dose CT puncture guidance method according to claim 14, wherein:
after the needle insertion is started, the needle is located at a certain depth inside the patient; and/or
the needle is straight; and/or
rigid registration comprises rotational registration and translational registration; and/or
the gantry of the triple-source and triple-detector is rotating or static.
